SEQUENCE LISTING (3 sequences)

SEQ ID NO: 1 - RNA, 100 nt, Artificial Sequence
  guide RNA
gccccgcagaucgauguagaguuuuagagcuagaaauagcaaguuaaaauaaggcuaguccguuaucaacuugaaaaaguggcaccgagucggugcuuuu

SEQ ID NO: 2 - DNA, 21 bp, Artificial Sequence
  UMPS-O-1 sequencing oligonucleotide
cccggggaaacccacgggtgc

SEQ ID NO: 3 - DNA, 23 bp, Artificial Sequence
  UMPS-O-2 sequencing oligonucleotide
agggtcggtctgcctgcttggct

(19) United States
(12) Patent Application Publication
(10) Pub. No.: US 2019/0269730 A1
(43) Pub. Date: Sep. 5, 2019

(54) REGULATABLE CELL LINES AND METHODS OF USE THEREOF

(71) Applicant: Auxolytic Ltd, London (GB)

(72) Inventor: James Patterson, London (GB)

(21) Appl. No.: 16/346,565

(22) PCT Filed: Nov. 1, 2017
(86) PCT No.: PCT/GB2017/053283

§ 371 Date: May 1, 2019

(30) Foreign Application Priority Data
Nov. 1, 2016 (GB) 1618414.5

(51) Int. Cl. A61K 35/17; C12N 15/113; C12N 15/90; C12N 9/22; C12N 15/52; C12N 5/0735; C12N 5/074; C12N 5/0789; C12N 5/0783; C12N 9/88; C12N 9/00

(52) U.S. Cl. CPC A61K 35/17; C12N 15/113; C12N 15/90; C12N 9/22; C12N 15/52; C12N 5/0606; C12Y 603/0401; C12N 5/0647; C12N 5/0636; C12N 9/88; C12N 9/93; C12Y 401/01023; C12N 5/0696

(57) ABSTRACT

The present invention provides regulatable cell lines with an engineered genome encoding an auxotrophic response system or element, and methods of use thereof. Growth and protein production of the cell lines is controlled by exposure of the cell line to an auxotrophic factor to activate the auxotrophic response system or element. Examples of auxotrophic factors include a nutrient, an enzyme, a moiety that alters pH, a moiety that alters temperature, a non-organic molecule, a non-essential amino acid, an altered concentration of a moiety, and a cellular niche environment. These regulatable cell lines may be used in regenerative medicine and enzyme replacement therapy.

### FIELD OF THE INVENTION

The invention relates to the generation of auxotrophic cell lines capable of growth only in the presence of a factor for which the cell lines are auxotrophic, or with the addition of an expression vector (chromosomally integrated or episomal) encoding an enzyme capable of synthesizing the auxotrophic factor.

### BACKGROUND OF THE INVENTION

Cell therapies have been shown to provide promising treatments. Yet, reintroduction of exogenously cultured cells into a human host carries risks. For example, chimeric antigen receptor T-cell (CAR-T) therapies carry risks of graft versus host disease (GvHD), and stem cell therapies carry risks of malignant transformation (e.g. teratoma).

In some cases, a transgene may be inserted as a safety switch. Transgene-based off switches also have a number of risks, such as (1) transgene insertion into a tumor suppressor leading to oncogenic transformation of the cell line, and (2) transgene insertion into an epigenetically silenced region leading to lack of expression and thus efficacy. Genome instability is a common phenotype in oncogenic transformation of a cell. Further, a point mutation or genetic loss of an exogenous suicide switch would be quickly selected for and amplified.

However, a safety switch based on targeting a signaling pathway of the cell depends on the physiology of the cell. For example, a cell that is in “pro-survival” mode may express caspase inhibitors, preventing cell death upon suicide switch induction.

Consequently, there is a long felt need in the art to develop cell therapies that are regulatable by factors outside of the subject being treated with a cell therapy and which have a decreased risk of oncogenic transformation or loss of efficacy in the patient.

### SUMMARY OF THE INVENTION

Various embodiments of the invention provide a regulatable cell line including an engineered genome, the engineered genome including or encoding an auxotrophic response system or element.

Certain embodiments provide the auxotrophic response system or element as responsive to the presence of an auxotrophic factor selected from the group consisting of a nutrient, an enzyme, altered pH, altered temperature, a non-organic molecule, a non-essential amino acid, an altered concentration of a moiety, and a niche environment. Certain embodiments provide the nutrient or the enzyme as neither toxic nor bioavailable in humans in sufficient concentrations to maintain the regulatable cell line. Typically such concentrations are measured compared to normal physiological conditions. For example, in one embodiment the nutrient is biotin, in another embodiment the nutrient is uracil. Certain embodiments provide the cell line as mammalian. For example, the mammalian cell line is human. Certain embodiments provide that the cell line includes at least one cell type selected from the group consisting of lymphocytes (such as T cells), induced pluripotent stem (iPS) cells, embryonic stem cells, somatic stem cells, haematopoetic stem cells and peripheral blood mononuclear cells (PBMCs). In certain embodiments, the engineered genome further includes or encodes a therapeutic product. Such a therapeutic product may be one that targets at least one therapeutic target selected from the group consisting of a cytokine, an antigen, a stem cell, and a T-cell. Certain aspects provide the engineered genome as including a knockout of a gene encoding a protein that produces or metabolizes the auxotrophic factor. Such genes may alternatively directly encode the auxotrophic factor.

Various embodiments of the invention provide a method of treating a disease, a disorder, or a condition in a subject with a regulatable cell line, the method including: (i) generating a cell line, e.g. a mammalian or human cell line, which is auxotrophic for a nutrient, an enzyme, an altered pH, an altered temperature, and/or a niche environment such that the nutrient, enzyme, altered pH, altered temperature, and niche environment is not present in the subject; (ii) contacting the subject with the resulting auxotrophic cell line of step (i); (iii) contacting the subject of (ii) with an auxotrophic factor which is selected from the nutrient, the enzyme, a moiety that alters pH, a moiety that alters temperature, and a cellular niche environment in the subject which was not previously present in the subject, such that the auxotrophic factor activates the auxotrophic system or element resulting in the growth of the cell line and/or the expression of one or more therapeutic entities for the subject. Use of a regulatable cell line according to the invention in treating a disease, a disorder, or a condition in a subject is also encompassed within the invention.

Certain embodiments provide the disease, the disorder, or the condition as selected from the group consisting of cancer, Parkinson's disease, graft versus host disease (GvHD), autoimmune conditions, hyperproliferative disorder or condition, malignant transformation, liver conditions, genetic conditions including inherited genetic defects, juvenile onset diabetes mellitus and ocular compartment conditions.

In certain embodiments, the disease, the disorder, or the condition affects at least one system of the body selected from the group consisting of muscular, skeletal, circulatory, nervous, lymphatic, respiratory endocrine, digestive, excretory, and reproductive systems. Certain embodiments provide the cell line as regenerative. In an aspect of the invention, the subject may be contacted with more than one regulatable cell line and/or with one or more auxotrophic factor. Certain embodiments provide localized release of the nutrient or the enzyme. For example, localized release is effected via utilization of a biocompatible device. In an aspect of the invention, the biocompatible device may restrict diffusion of the cell line in the subject. Certain embodiments of the method provide removing the auxotrophic factor to deplete therapeutic effects of the regulatable cell line in the subject or to induce cell death in the regulatable cell line. Certain embodiments of the method provide the therapeutic effects as including at least one selected from the group consisting of: molecule trafficking, inducing cell death, cell death, and recruiting of additional cells. Certain embodiments of the method provide that the cell line is derived from the subject prior to generating an auxotrophic response or element in the cell line.

Also provided herein are methods of making the auxotrophic cell lines of the invention. Further provided are methods of treating diseases, disorders or conditions in a subject comprising: (a) administering to the subject a regulatable cell line according to the invention, and (b) administering the auxotrophic factor to the subject in an amount sufficient to promote growth of the regulatable human cell line. Also provided is a nutrient for use in administration to a patient comprising a regulatable cell line according to the invention. In certain embodiments, the regulatable cell line is derived from cells from the subject, for example, by isolating cells from the subject, and engineering the genome of said cells to comprise or encode an auxotrophic response system, e.g. by knocking out or down regulating expression or activity of a gene in said cells thereby causing auxotrophy.

### DETAILED DESCRIPTION OF THE INVENTION

### I. Introduction

Biological containment is a genetic technique that allows for regulation of cell proliferation, transcription, and translation. See, Kato et al., Peer J, 3:e1247; DOI 10.7717/peerj.1247 (2015). Containment may be achieved by genetically engineering a cell line to eliminate an essential gene to cause the cell line to become auxotrophic. An auxotrophy is a condition of a cell that causes an inability of the cell to produce an organic compound required for growth and reproduction. In certain embodiments, an auxotrophy may be induced in a cell by genetically engineering the genome to encode an auxotrophic response system or element. As used herein, the term “auxotrophic response system or element” refers to a portion of a genome of the regulatable cell lines described herein that is responsive to the presence of an auxotrophic factor. This regulation is driven by either a knockout of a gene or the downregulation of a gene's activity, such that the cell lines become auxotrophic for a specific factor.

The auxotrophic response system or element may be responsive to the presence of an auxotrophic factor such as a nutrient, an enzyme, an altered pH, an altered temperature, a non-organic molecule, a non-essential amino acid, an altered concentration of a moiety, and a niche environment. In certain embodiments, the auxotrophic factor is a nutrient or the enzyme that is neither toxic nor bioavailable in the subject in concentrations sufficient to sustain the regulatable cell line. Typically such concentrations are measured compared to normal physiological conditions. The term “niche environment” as used herein, refers to a habitat for the regulatable cell line described herein that supplies the factors necessary for proliferation or production of a therapeutic entity.

Outside of the controlled area, the cell lines should die because of a lack of the auxotrophic factor or presence of the auxotrophic factor at too low a concentration to activate the auxotrophic response system or element, which decreases the risks associated with other cell-based therapies that include oncogenic transformation.

### II. Compositions of the Invention

As discussed above, cell lines that have been genetically engineered to include an auxotrophic response system or element that is activated by a factor that is externally controlled allows for selective proliferation and production of therapeutic entities that are encoded by the genome of the regulatable cell line. This approach may be useful to overcome many of the problems associated with cell-based therapies.

Compositions of the invention herein include cell lines that are genetically engineered to include an auxotrophic response system or element. A “cell line” as used herein, refers to a population of cells descended from the same cell, with each cell of the population having a similar genetic make-up.

In certain embodiments, the cell line that is genetically engineered to include an auxotrophic response system or element may be mammalian. Specifically, the mammalian cell line may be human. Alternatively, the cell line may not be mammalian.

In certain embodiments, the auxotrophic response system or element may be a gene knockout. As used herein, a “gene knockout cell line” is a cell line with a targeted disruption of a gene resulting in complete loss of function that has been achieved using a molecular biology techniques known in the art. Alternatively, genes may be cloned with an endogenous promoter, or a commonly used strong/weak constitutive/inducible promoters (SV40/CMV/tet etc.) and inserted into the genome of the cell line. For example, a “knock-in” regulatable cell lines.

Auxotrophic human cell lines provide a much-needed increase in the number of positive selection markers available for genome editing of human/mammalian cells.

Various methods are known in the art for editing nucleic acid, for example to cause a gene knockout or expression of a gene to be downregulated. For example, various nuclease systems, such as zinc finger nucleases (ZFN), transcription activator-like effector nucleases (TALEN), meganucleases, or combinations thereof are known in the art to be used to edit nucleic acid and may be used in the present invention. In recent times, the clustered regularly interspersed short palindromic repeats (CRISPR)/CRISPR-associated (Cas) (CRISPR/Cas) nuclease system has become more commonly used for genome engineering. The CRISPR/Cas system is detailed in, for example WO2013/176772, WO2014/093635 and WO2014/089290. Its use in T-cells is suggested in WO2014/191518.

The time-limiting factor for generation of mutant (knock-out, knock-in, or gene replaced) cell lines was the clone screening and selection before development of the CRISPR/Cas9 platform. The term “CRISPR/Cas9 platform” as used herein, refers to a genetic engineering tool that includes a guide RNA (gRNA) sequence with a binding site for Cas9 and a targeting sequence specific for the area to be modified. The Cas9 binds the gRNA to form a ribonucleoprotein that binds and cleaves the target area. Before CRISPR/Cas9, mammalian genome editing could be multiplexed, but selection for particular mutations, transgene insertions, or gene deletions required antibiotic resistance markers or laborious PCR based screening methods.

In addition to the CRISPR/Cas 9 platform (which is a type II CRISPR/Cas system), alternative systems exist including type I CRISPR/Cas systems, type III CRISPR/Cas systems, and type V CRISPR/Cas systems. Various CRISPR/Cas9 systems have been disclosed, including Streptococcus pyogenes Cas9 (SpCas9), Streptococcus thermophilus Cas9 (StCas9), Campylobacter jejuni Cas9 (CjCas9) and Neisseria cinerea Cas9 (NcCas9) to name a few. Alternatives to the Cas system include the Francisella novicida Cpf1 (FnCpf1), Acidaminococcus sp. Cpf1 (AsCpf1), and Lachnospiraceae bacterium ND2006 Cpf1 (LbCpf1) systems. Any of the above CRISPR systems may be used in methods to generate the cell lines of the invention. In one embodiment, the CRISPR system used is the CRISPR/Cas9 system. In one embodiment, the S. pyogenes CRISPR/Cas9 system is used.

Target genes may be edited, for example using the above methods, by deleting, inserting or substituting one or more nucleotides within said target gene, leading to the knockout of that gene, or the downregulation of expression of that gene.

Currently, only five antibiotics are commonly used for mammalian cell positive selection: blasticidin, G418, hygromycin, puromycin, and zeocin. Additionally, heterogeneity in the response of different cell lines to these antibiotics makes selection a laborious process. Thus, alternative selection methods are desired.

Therapeutic entities encoded by the genome of the regulatable cell line may cause therapeutic effects, such as molecule trafficking, inducing cell death, recruitment of additional cells, or cell growth.

### Regulatable Cell Lines

To prevent immune rejection of the cells when administered to a subject, the regulatable cell line may be derived from the subject's own cells. For example, the cells may be stem cells isolated from the subject for use in a regenerative medical treatment in any of epithelium, cartilage, bone, smooth muscle, striated muscle, neural epithelium, stratified squamous epithelium, and ganglia. Disease that results from the death or dysfunction of one or a few cell types, such as Parkinson's disease and juvenile onset diabetes, are also commonly treated using stem cells. See, Thomson et al., Science, 282:1145-1147 (1998).

The regulatable cell lines described herein allow for a treatment to be controlled by conditions external to the cellular environment through an auxotrophic response system or element. The auxotrophic response system or element may include a gene knockout or a downregulation of expression. In certain embodiments, the auxotrophic response system or element is a gene knockout of the auxotrophic factor or a gene that affects metabolism of the auxotrophic factor.

Gene candidates for knockout to develop an exemplary regulatable cell line including a genetically engineered genome were identified from various sources, including the following: (1) never mutated genes with metabolic process annotations in The Cancer Genome Atlas (TCGA) from the National Institutes of Health (NIH), (2) identify human homologues of commonly used gene deletions in model organisms, (3) published human essential gene screens (culture media may contain supplements for the synthase, and (4) enzymes mutated in human hereditary metabolic diseases. Alternatively, the cell lines were purchased from a commercial vendor with gene knockouts already made, such as the HAP1 cell line from Horizon® Discovery Group, PLC. The knockout in the genetically engineered genome may be of a gene that expresses the auxotrophic factor.

Various genes may be knocked out to create a regulatable cell line of the invention. In one embodiment, the gene that is knocked out or downregulated is selected from uridine monophosphate synthetase (UMPS) (creating a cell line auxotrophic for uracil) and holocarboxylase synthetase (creating a cell line auxotrophic for biotin). In one embodiment, the gene that is knocked out or downregulated is uridine monophosphate synthetase. Table 1 shows a more complete list of genes that may be knocked out and the auxotrophic factor required to restore cell growth.

TABLE 1

| Gene | Auxotrophic factor | Gene | Auxotrophic factor | Gene | Auxotrophic factor |
|---|---|---|---|---|---|
| UMPS | Uracil | HOXA9 | adenine | ISCA1 | lysine |
| HLCS | Biotin | HOXB1 | adenine | ISCA1P1 | lysine |
| COQ6 | Nicotinic acid | HOXB13 | adenine | ISCA2 | lysine |
| KMO | Nicotinic acid | HOXB2 | adenine | DECR1 | ergosterol |
| MITF | glutamate(1−) | HOXB3 | adenine | HSD17B12 | ergosterol |
| MLX | glutamate(1−) | HOXB4 | adenine | HSD17B3 | ergosterol |
| TFE3 | glutamate(1−) | HOXB5 | adenine | HSD17B7 | ergosterol |
| TFEB | glutamate(1−) | HOXB6 | adenine | HSD17B7P2 | ergosterol |
| TFEC | glutamate(1−) | HOXB7 | adenine | HSDL1 | ergosterol |
| USF1 | glutamate(1−) | HOXB8 | adenine | HSDL2 | ergosterol |
| USF2 | glutamate(1−) | HOXB9 | adenine | SMS | 0.25 mM spermine |
| AACS | Lysine | HOXC10 | adenine | SRM | 0.25 mM spermine |
| ACSS3 | Lysine | HOXC11 | adenine | KYNU | Nicotinic acid |
| DHRS7 | Lysine | HOXC12 | adenine | ACO2 | Glutamic acid |
| DHRS7B | Lysine | HOXC13 | adenine | ADSL | Adenine |
| DHRS7C | Lysine | HOXC4 | adenine | FH | adenine |
| HSD11B1 | Lysine | HOXC5 | adenine | VPS33A | methionine |
| HSD11B1L | Lysine | HOXC6 | adenine | VPS33B | methionine |
| KDSR | Lysine | HOXC8 | adenine | VPS36 | ethanolamine |
| RDH8 | Lysine | HOXC9 | adenine | UMPS | uracil |
| MTHFR | methionine | HOXD1 | adenine | HMGCS1 | ergosterol |
| DUT | dTMP | HOXD10 | adenine | HMGCS2 | ergosterol |
| THNSL1 | threonine | HOXD11 | adenine | IL4I1 | 0.1 mM beta-alanine |
| THNSL2 | threonine | HOXD12 | adenine | KDM1B | 0.1 mM beta-alanine |
| CTH | Cysteine | HOXD13 | adenine | PAOX | 0.1 mM beta-alanine |
| SLC7A10 | L-arginine | HOXD3 | adenine | SMOX | 0.1 mM beta-alanine |
| SLC7A11 | L-arginine | HOXD4 | adenine | CCS | methionine, lysine |
| SLC7A13 | L-arginine | HOXD8 | adenine | ILVBL | valine, isoleucine |
| SLC7A5 | L-arginine | HOXD9 | adenine | ALDH1A1 | Pantothenic acid |
| SLC7A6 | L-arginine | PDX1 | adenine | ALDH1A2 | Pantothenic acid |
| SLC7A7 | L-arginine | HMGCL | lysine | ALDH1A3 | Pantothenic acid |
| SLC7A8 | L-arginine | HMGCLL1 | lysine | ALDH1B1 | Pantothenic acid |
| SLC7A9 | L-arginine | HMBS | heme | ALDH2 | Pantothenic acid |
| MPI | D-mannose | UROD | heme | MVK | ergosterol |
| PYCR1 | proline | IP6K1 | arginine | GMPS | guanine |
| PYCR2 | proline | IP6K2 | arginine | PPAT | adenine |
| PYCRL | proline | IP6K3 | arginine | SLC25A26 | biotin |
| CYP51A1 | ergosterol | IPMK | arginine | ATF4 | methionine |
| ASL | arginine | ALAS1 | cysteine | ATF5 | methionine |
| FH | arginine | ALAS2 | cysteine | HMGCL | leucine |
| LSS | ergosterol | GCAT | cysteine | HMGCLL1 | leucine |
| FDFT1 | ergosterol | ACO1 | leucine | ADSS | adenine |
| BCAT1 | valine, leucine | ACO2 | leucine | ADSSL1 | adenine |
| BCAT2 | valine, leucine | IREB2 | leucine | G6PD | methionine |
| HRSP12 | isoleucine | ALAD | cysteine | H6PD | methionine |
| AADAT | histidine | SCD | oleicacid | AMD1 | 0.25 mM spermine |
| ACCS | histidine | SCD5 | oleicacid | ASS1 | arginine |
| ACCSL | histidine | MPC1 | valine, leucine | RPUSD2 | riboflavin |
| CCBL1 | histidine | MPC1L | valine, leucine | MITF | glutamate(1−) |
| CCBL2 | histidine | MTHFR | methionine | TFE3 | glutamate(1−) |
| GPT | histidine | AASDHPPT | lysine | TFEB | glutamate(1−) |
| GPT2 | histidine | CH25H | ergosterol | TFEC | glutamate(1−) |
| TAT | histidine | FAXDC2 | ergosterol | USF1 | glutamate(1−) |
| MMS19 | methionine | MSMO1 | ergosterol | USF2 | glutamate(1−) |
| CEBPA | methionine | PFAS | adenine | PAICS | adenine |
| CEBPB | methionine | SLC25A19 | thiamine | PSAT1 | serine |
| CEBPD | methionine | ETFDH | thiamine(1+) | DHFR | dTMP |
| CEBPE | methionine | CBS | cysteine | DHFRL1 | dTMP |
| CEBPG | methionine | CBSL | cysteine | FPGS | methionine |
| AASS | Lysine | SQLE | ergosterol | UROS | heme |
| PAPSS1 | methionine | MTHFD1 | adenine | CAD | arginine |
| PAPSS2 | methionine | MTHFD1L | adenine | CPS1 | arginine |
| GFPT1 | D-glucosamine | MTHFD2 | adenine | OTC | arginine |
| GFPT2 | D-glucosamine | MTHFD2L | adenine | ACAT1 | ergosterol |
| SLC25A34 | leucine | PSPH | serine | GLRX5 | Glutamic acid |
| SLC25A35 | leucine | MARS | methionine | PLCB1 | ornithine |
| AZIN1 | 0.25 mM putrescine | MARS2 | methionine | PLCB2 | ornithine |
| AZIN2 | 0.25 mM putrescine | GCH1 | 5- | PLCB3 | ornithine |
| | | | formyltetrahydrofolic | | |
| | | | acid | | |
| ODC1 | 0.25 mM putrescine | NAGS | arginine | PLCB4 | ornithine |
| DHODH | Uracil | OTC | arginine | PLCD1 | ornithine |
| DPYD | Uracil | GCLC | glutathione | PLCD3 | ornithine |
| FDXR | Uracil | CAD | uracil | PLCD4 | ornithine |
| MTHFD2 | adenine | FDPS | ergosterol | PLCE1 | ornithine |
| MTHFD2L | adenine | PM20D1 | leucine | PLCG1 | ornithine |
| MYB | adenine | PIN1 | galactose | PLCG2 | ornithine |
| MYBL1 | adenine | HAAO | nicotinicacid | PLCH1 | ornithine |
| MYBL2 | adenine | MAX | methionine | PLCH2 | ornithine |
| SNAPC4 | adenine | MITF | methionine | PLCL1 | ornithine |
| MTRR | methionine | TFE3 | methionine | PLCL2 | ornithine |
| QPRT | Nicotinic acid | TFEB | methionine | PLCZ1 | ornithine |
| GSX2 | adenine | TFEC | methionine | GLUL | glutamine |
| HOXA1 | adenine | USF1 | methionine | LGSN | glutamine |
| HOXA10 | adenine | USF2 | methionine | ISCA1 | lysine |
| HOXA11 | adenine | IDO1 | Nicotinic acid | ISCA2 | lysine |
| HOXA13 | adenine | IDO2 | Nicotinic acid | SMS | 10uM spermidine |
| HOXA2 | adenine | CCS | methionine | SRM | 10uM spermidine |
| HOXA3 | adenine | SOD1 | methionine | PDHB | tryptophan |
| HOXA4 | adenine | SOD3 | methionine | TKT | tryptophan |
| HOXA5 | adenine | CAD | arginine | TKTL1 | tryptophan |
| HOXA6 | adenine | CPS1 | arginine | TKTL2 | tryptophan |
| HOXA7 | adenine | IBA57 | glutamicacid | KATNA1 | ethanolamine |
| KATNAL1 | ethanolamine | VPS4A | ethanolamine | PAICS | Adenine |
| KATNAL2 | ethanolamine | VPS4B | ethanolamine | | |

The genes of Table 1 were collated by selecting S. cerevisiae genes with a phenotype annotated as “Auxotrophy” downloaded with “Chemical” data from the yeast phenotype ontology database on the Saccharomyces genome database (SGD) (Cherry et al. 2012, Nucleic Acids Res. 40:D700-D705). These genes were converted into human homologues using YeastMine (also found on the SGD).

The cell line may be selected from lymphocytes, induced pluripotent stem (iPS) cells, embryonic stem cells, somatic stem cells, haematopoetic stem cells or peripheral blood mononuclear cells (PBMCs). In one embodiment the cell line is a T cell line. In one embodiment the cell line is a chimeric antigen receptor (CAR)-T cell line. In addition to the auxotrophic response system or element, a therapeutic product may be introduced to the cell line by genetically engineering the genome to encode a therapeutic product. Such a therapeutic product may target at least one of a cytokine, an antigen, or a stem cell.

In certain embodiments, an auxotrophic response system or element includes a translational switch that is activated by the auxotrophic factor. The switch may regulate target gene transcripts with a stop codon inserted next to the start codon of the transcript. In the presence of the auxotrophic factor, translation continues through the stop codon leading to expression of the target gene. In certain embodiments, absence of the auxotrophic factor results in termination of translation at the stop codon, and a lack of expression of the target gene. Rate of expression of a therapeutic entity may also be regulated by adjusting the concentration of the auxotrophic factor. See, Chaveroux et al., Nature Biotechnology, 34, 746-751 (2016). In one embodiment of the invention, the regulatable cell line does not comprise an exogenous inducible promoter.

An auxotrophy-based safety mechanism circumvents many of the risks to patients associated with current cell therapies. By supplementing a patient with a defined auxotrophic factor during the course of the therapy, and removing the factor upon therapy cessation or some other safety based indication, cell growth is physically limited. If the cell does not contain a necessary enzyme and does not have a nutrient necessary for biosynthesis, then the cell stops dividing and does not have a self-evident mechanism for the development of resistance. By manipulating levels of the auxotrophic factor, the growth rate of cells in vivo is controlled. Multiple cell lines may be controlled independently in vivo by using separate auxotrophies. Location specific growth may be controlled by localized nutrient release, such as exogenously grown pancreatic B cells administered within a biocompatible device that releases a nutrient and prevents cell escape. For example, the present invention may be used in conjunction with CAR-T cell technology, to allow more defined control over the activity of CAR-T cells in vivo. As noted above, CRISPR engineering of T cells is discussed in EP3004349.

The cell lines may include stem cells that were maintained and differentiated using the techniques below as shown in U.S. Pat. No. 8,945,862, which is hereby incorporated by reference in its entirety. In one embodiment, the stem cell is not a human embryonic stem cell. Furthermore, the cell lines may include stem cells made by the techniques disclosed in WO2003/046141 or Chung et al. (Cell Stem Cell, February 2008, Vol. 2, pages 113-117).

The genetically engineered genome of the cell line may encode a therapeutic product. Such a therapeutic product may be one that targets at least one of a cytokine, an antigen, and a stem cell.

### Auxotrophic Factors

The auxotrophic response system or element of the regulatable cell line may be responsive to the presence of an auxotrophic factor selected from a nutrient, an enzyme, an altered pH, an altered temperature, a non-organic molecule, a non-essential amino acid, an altered concentration of a moiety, and a niche environment. The auxotrophic factor may not be toxic, bioavailable, or present in a sufficient concentration to activate the auxotrophic response system or element in a subject that is treated with the regulatable cell line. Typically such concentrations are measured compared to normal physiological conditions.

For example, the auxotrophic factor may be a nutrient that is a substance required for proliferation or that functions as a cofactor in metabolism of the regulatable cell line. Various auxotrophic factors are disclosed in Table 1. In certain embodiments, the auxotrophic factor is selected from biotin, alanine, aspartate, asparagine, glutamate, serine, uracil and cholesterol. Biotin, also known as vitamin B7, is necessary for cell growth. In one embodiment of the invention the regulatable cell line is auxotrophic for biotin or uracil.

Alternatively, the auxotrophic factor is an enzyme that catalyzes cellular metabolic processes and/or expression of a therapeutic product that is encoded by the genome of the regulatable cell line. The enzyme may be selected from acid alpha-glucosidase, lysosomal glucocerebrosidase, and globotriaosylceramide.

### III. Pharmaceutical Compositions

The regulatable cell line may be administered directly to a subject or in combination with an auxotrophic factor. Although the descriptions of pharmaceutical compositions provided herein are principally directed to pharmaceutical compositions which are suitable for administration to humans, it will be understood by the skilled artisan that such compositions are generally suitable for administration to any other animal, e.g., to non-human animals or non-human mammals. Modification of pharmaceutical compositions suitable for administration to humans in order to render the compositions suitable for administration to various animals is well understood, and the ordinarily skilled veterinary pharmacologist can design and/or perform such modification with merely ordinary, if any, experimentation. Subjects to which administration of the pharmaceutical compositions is contemplated include, but are not limited to, humans and/or other primates; mammals, including commercially relevant mammals such as cattle, pigs, horses, sheep, cats, dogs, mice, rats, birds, including commercially relevant birds such as poultry, chickens, ducks, geese, and/or turkeys. In some embodiments, compositions are administered to humans, human patients, or subjects.

The pharmaceutical compositions described herein may be used in a method of treating a disease, a disorder, or a condition in a subject, the method including: (i) generating a cell line which is auxotrophic for a nutrient, an enzyme, an altered pH, an altered temperature, an altered concentration of a moiety, and/or a niche environment, such that the nutrient, enzyme, altered pH, altered temperature, and niche environment is not present in the subject, (ii) contacting the subject with the resulting auxotrophic cell line of step (i); (iii) contacting the subject of (ii) with the auxotrophic factor which is selected from the nutrient, enzyme, moiety that alters pH and/or temperature, and a cellular niche environment in the subject, such that the auxotrophic factor activates the auxotrophic system or element resulting in the growth of the cell line and/or the expression of one or more therapeutic entities for the subject.

The pharmaceutical compositions of the invention may also be used in a method of treating a disease, a disorder, or a condition in a subject, comprising (a) administering to the subject a regulatable cell line according to the invention, and (b) administering the auxotrophic factor to the subject in an amount sufficient to promote growth of the regulatable cell line.

Compositions comprising a nutrient auxotrophic factor may also be used for administration to a human comprising a regulatable cell line of the invention.

Diseases, disorders, or conditions affects at least one system of the body selected from the group consisting of muscular, skeletal, circulatory, nervous, lymphatic, respiratory endocrine, digestive, excretory, and reproductive systems. For example, the disease, disorder, or condition that may be treated by the pharmaceutical compositions described herein is selected from the group of cancer, Parkinson's disease, graft versus host disease (GvHD), autoimmune conditions, hyperproliferative disorder or condition, malignant transformation, liver conditions, genetic conditions, juvenile onset diabetes mellitus, and ocular compartment conditions. Conditions that affect more than one cell type in the subject may be treated with more than one regulatable cell line with each cell line activated by a different auxotrophic factor. In some cases, a subject may be administered more than one auxotrophic factor.

At the conclusion of treatment, the auxotrophic factor may be removed to deplete therapeutic effects of the regulatable cell line in the subject or to induce cell death in the regulatable cell line.

### IV. Formulations

### Cellular Engineering Formulations

The regulatable cell line is genetically engineered to include an auxotrophic response system or element. Delivery of Cas9 protein/gRNA ribonucleoprotein complexes (Cas9 RNPs) into the cell line may be performed by liposome-mediated transfection, electroporation, or nuclear localization to produce the indels resulting in an auxotrophic response system or element in the cell line.

### Therapeutic Formulations

The regulatable cell line or auxotrophic factor of the invention may be formulated using one or more excipients to: (1) increase stability; (2) alter the biodistribution (e.g., target the cell line to specific tissues or cell types); (3) alter the release profile of an encoded therapeutic product; and/or (4) improve uptake of the auxotrophic factor.

Formulations of the present invention can include, without limitation, saline, liposomes, lipid nanoparticles, polymers, peptides, proteins, and combinations thereof.

Formulations of the pharmaceutical compositions described herein may be prepared by any method known or hereafter developed in the art of pharmacology. As used herein the term “pharmaceutical composition” refers to compositions including at least one active ingredient and optionally one or more pharmaceutically acceptable excipients. Pharmaceutical compositions of the invention may be sterile.

In general, such preparatory methods include the step of associating the active ingredient with an excipient and/or one or more other accessory ingredients. As used herein, the phrase “active ingredient” generally refers to a regulatable cell line including an engineered genome encoding an auxotrophic response system or element as described herein.

Formulations of the regulatable cell line or the auxotrophic factor and pharmaceutical compositions described herein may be prepared by any method known or hereafter developed in the art of pharmacology. In general, such preparatory methods include the step of bringing the active ingredient into association with an excipient and/or one or more other accessory ingredients, and then, if necessary and/or desirable, dividing, shaping, and/or packaging the product into a desired single- or multi-dose unit.

A pharmaceutical composition in accordance with the present disclosure may be prepared, packaged, and/or sold in bulk, as a single unit dose, and/or as a plurality of single unit doses. As used herein, a “unit dose” refers to a discrete amount of the pharmaceutical composition including a predetermined amount of the active ingredient. The amount of the active ingredient is generally equal to the dosage of the active ingredient which would be administered to a subject and/or a convenient fraction of such a dosage such as, for example, one-half or one-third of such a dosage.

Relative amounts of the active ingredient (e.g. the regulatable cell line or auxotrophic factor), a pharmaceutically acceptable excipient, and/or any additional ingredients in a pharmaceutical composition in accordance with the present disclosure may vary, depending upon the identity, size, and/or condition of the subject being treated and further depending upon the route by which the composition is to be administered. For example, the composition may include between 0.1% and 99% (w/w) of the active ingredient. By way of example, the composition may include between 0.1% and 100%, e.g., between 0.5 and 50%, between 1-30%, between 5-80%, or at least 80% (w/w) active ingredient.

### Excipients and Diluents

In some embodiments, a pharmaceutically acceptable excipient may be at least 95%, at least 96%, at least 97%, at least 98%, at least 99%, or 100% pure. In some embodiments, an excipient is approved for use for humans and for veterinary use. In some embodiments, an excipient may be approved by United States Food and Drug Administration. In some embodiments, an excipient may be of pharmaceutical grade. In some embodiments, an excipient may meet the standards of the United States Pharmacopoeia (USP), the European Pharmacopoeia (EP), the British Pharmacopoeia, and/or the International Pharmacopoeia.

Excipients, as used herein, include, but are not limited to, any and all solvents, dispersion media, diluents, or other liquid vehicles, dispersion or suspension aids, surface active agents, isotonic agents, thickening or emulsifying agents, preservatives, and the like, as suited to the particular dosage form desired. Various excipients for formulating pharmaceutical compositions and techniques for preparing the composition are known in the art (see Remington: The Science and Practice of Pharmacy, 21st Edition, A. R. Gennaro, Lippincott, Williams & Wilkins, Baltimore, Md., 2006; incorporated herein by reference in its entirety). The use of a conventional excipient medium may be contemplated within the scope of the present disclosure, except insofar as any conventional excipient medium may be incompatible with a substance or its derivatives, such as by producing any undesirable biological effect or otherwise interacting in a deleterious manner with any other component(s) of the pharmaceutical composition.

Exemplary diluents include, but are not limited to, calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, etc., and/or combinations thereof.

### Inactive Ingredients

In some embodiments, regulatable cell formulations may include at least one inactive ingredient. As used herein, the term “inactive ingredient” refers to one or more agents that do not contribute to the activity of the active ingredient of the pharmaceutical composition included in formulations. In some embodiments, all, none or some of the inactive ingredients which may be used in the formulations of the present invention may be approved by the U.S. Food and Drug Administration (FDA).

### Pharmaceutically Acceptable Salts

Formulations of the invention may also include one or more pharmaceutically acceptable salts. As used herein, “pharmaceutically acceptable salts” refers to derivatives of the disclosed compounds such that the parent compound is modified by converting an existing acid or base moiety to its salt form (e.g., by reacting the free base group with a suitable organic acid). Examples of pharmaceutically acceptable salts include, but are not limited to, mineral or organic acid salts of basic residues such as amines; alkali or organic salts of acidic residues such as carboxylic acids; and the like. Representative acid addition salts include acetate, acetic acid, adipate, alginate, ascorbate, aspartate, benzenesulfonate, benzene sulfonic acid, benzoate, bisulfate, borate, butyrate, camphorate, camphorsulfonate, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, valerate salts, and the like. Representative alkali or alkaline earth metal salts include sodium, lithium, potassium, calcium, magnesium, and the like, as well as nontoxic ammonium, quaternary ammonium, and amine cations, including, but not limited to ammonium, tetramethylammonium, tetraethylammonium, methylamine, dimethylamine, trimethylamine, triethylamine, ethylamine, and the like. The pharmaceutically acceptable salts of the present disclosure include the conventional non-toxic salts of the parent compound formed, for example, from non-toxic inorganic or organic acids.

Solvates may be prepared by crystallization, recrystallization, or precipitation from a solution that includes organic solvents, water, or a mixture thereof. Examples of suitable solvents are ethanol, water (for example, mono-, di-, and tri-hydrates), N-methylpyrrolidinone (NMP), dimethyl sulfoxide (DMSO), N,N′-dimethylformamide (DMF), N,N′-dimethylacetamide (DMAC), 1,3-dimethyl-2-imidazolidinone (DMEU), 1,3-dimethyl-3,4,5,6-tetrahydro-2-(1H)-pyrimidinone (DMPU), acetonitrile (ACN), propylene glycol, ethyl acetate, benzyl alcohol, 2-pyrrolidone, benzyl benzoate, and the like. When water is the solvent, the solvate is referred to as a “hydrate.”

### V. Dosing and Administration

The regulatable cell lines or auxotrophic factors of the present invention included in the pharmaceutical compositions described above may be administered by any delivery route which results in a therapeutically effective outcome. These include, but are not limited to, enteral (into the intestine), gastroenteral, epidural (into the dura mater), oral (by way of the mouth), transdermal, intracerebral (into the cerebrum), intracerebroventricular (into the cerebral ventricles), epicutaneous (application onto the skin), intradermal (into the skin itself), subcutaneous (under the skin), nasal administration (through the nose), intravenous (into a vein), intravenous bolus, intravenous drip, intra-arterial (into an artery), intramuscular (into a muscle), intracardiac (into the heart), intraosseous infusion (into the bone marrow), intrathecal (into the spinal canal), intraparenchymal (into brain tissue), intraperitoneal (infusion or injection into the peritoneum), intravesical infusion, intravitreal, (through the eye), intracavernous injection (into a pathologic cavity), intracavitary (into the base of the penis), intravaginal administration, intrauterine, extra-amniotic administration, transdermal (diffusion through the intact skin for systemic distribution), transmucosal (diffusion through a mucous membrane), transvaginal, insufflation (snorting), sublingual, sublabial, enema, eye drops (onto the conjunctiva), or in ear drops, auricular (in or by way of the ear), buccal (directed toward the cheek), conjunctival, cutaneous, dental (to a tooth or teeth), electro-osmosis, endocervical, endosinusial, endotracheal, extracorporeal, hemodialysis, infiltration, interstitial, intra-abdominal, intra-amniotic, intra-articular, intrabiliary, intrabronchial, intrabursal, intracartilaginous (within a cartilage), intracaudal (within the cauda equine), intracisternal (within the cisterna magna cerebellomedularis), intracorneal (within the cornea), dental intracornal, intracoronary (within the coronary arteries), intracorporus cavernosum (within the dilatable spaces of the corporus cavernosa of the penis), intradiscal (within a disc), intraductal (within a duct of a gland), intraduodenal (within the duodenum), intradural (within or beneath the dura), intraepidermal (to the epidermis), intraesophageal (to the esophagus), intragastric (within the stomach), intragingival (within the gingivae), intraileal (within the distal portion of the small intestine), intralesional (within or introduced directly to a localized lesion), intraluminal (within a lumen of a tube), intralymphatic (within the lymph), intramedullary (within the marrow cavity of a bone), intrameningeal (within the meninges), intramyocardial (within the myocardium), intraocular (within the eye), intraovarian (within the ovary), intrapericardial (within the pericardium), intrapleural (within the pleura), intraprostatic (within the prostate gland), intrapulmonary (within the lungs or its bronchi), intrasinal (within the nasal or periorbital sinuses), intraspinal (within the vertebral column), intrasynovial (within the synovial cavity of a joint), intratendinous (within a tendon), intratesticular (within the testicle), intrathecal (within the cerebrospinal fluid at any level of the cerebrospinal axis), intrathoracic (within the thorax), intratubular (within the tubules of an organ), intratumor (within a tumor), intratympanic (within the aurus media), intravascular (within a vessel or vessels), intraventricular (within a ventricle), iontophoresis (by means of electric current where ions of soluble salts migrate into the tissues of the body), irrigation (to bathe or flush open wounds or body cavities), laryngeal (directly upon the larynx), nasogastric (through the nose and into the stomach), occlusive dressing technique (topical route administration which is then covered by a dressing which occludes the area), ophthalmic (to the external eye), oropharyngeal (directly to the mouth and pharynx), parenteral, percutaneous, periarticular, peridural, perineural, periodontal, rectal, respiratory (within the respiratory tract by inhaling orally or nasally for local or systemic effect), retrobulbar (behind the pons or behind the eyeball), soft tissue, subarachnoid, subconjunctival, submucosal, topical, transplacental (through or across the placenta), transtracheal (through the wall of the trachea), transtympanic (across or through the tympanic cavity), ureteral (to the ureter), urethral (to the urethra), vaginal, caudal block, diagnostic, nerve block, biliary perfusion, cardiac perfusion, photopheresis, and spinal.

### Parenteral and Injectable Administration

In some embodiments, pharmaceutical compositions including the regulatable cell line or auxotrophic factor of the present invention may be administered parenterally. Liquid dosage forms for oral and parenteral administration include, but are not limited to, pharmaceutically acceptable emulsions, microemulsions, solutions, suspensions, syrups, and/or elixirs. In addition to active ingredients, liquid dosage forms may include inert diluents commonly used in the art such as, for example, water or other solvents, solubilizing agents, and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, oral compositions may include adjuvants such as wetting agents, emulsifying and suspending agents, sweetening, flavoring, and/or perfuming agents. In certain embodiments for parenteral administration, compositions are mixed with solubilizing agents such as CREMOPHOR®, alcohols, oils, modified oils, glycols, polysorbates, cyclodextrins, polymers, and/or combinations thereof. In other embodiments, surfactants are included such as hydroxypropylcellulose.

Injectable preparations, for example, sterile injectable aqueous or oleaginous suspensions may be formulated according to the known art using suitable dispersing agents, wetting agents, and/or suspending agents. Sterile injectable preparations may be sterile injectable solutions, suspensions, and/or emulsions in nontoxic parenterally acceptable diluents and/or solvents, for example, as a solution in 1,3-butanediol. Among the acceptable vehicles and solvents that may be employed are water, Ringer's solution, U.S.P., and isotonic sodium chloride solution. Sterile, fixed oils are conventionally employed as a solvent or suspending medium. For this purpose, any bland fixed oil can be employed including synthetic mono- or diglycerides. Fatty acids such as oleic acid can be used in the preparation of injectables.

Injectable formulations may be sterilized, for example, by filtration through a bacterial-retaining filter, and/or by incorporating sterilizing agents in the form of sterile solid compositions which can be dissolved or dispersed in sterile water or other sterile injectable medium prior to use.

In order to prolong the effect of active ingredients, it is often desirable to slow the absorption of active ingredients from subcutaneous or intramuscular injections. This may be accomplished by the use of liquid suspensions of crystalline or amorphous material with poor water solubility. The rate of absorption of active ingredients depends upon the rate of dissolution which, in turn, may depend upon crystal size and crystalline form. Alternatively, delayed absorption of a parenterally administered drug form is accomplished by dissolving or suspending the drug in an oil vehicle. Injectable depot forms are made by forming microencapsule matrices of the drug in biodegradable polymers such as polylactide-polyglycolide. Depending upon the ratio of drug to polymer and the nature of the particular polymer employed, the rate of drug release can be controlled. Examples of other biodegradable polymers include poly(orthoesters) and poly(anhydrides). Depot injectable formulations are prepared by entrapping the drug in liposomes or microemulsions which are compatible with body tissues.

### Rectal and Vaginal Administration

In some embodiments, pharmaceutical compositions including the regulatable cell line or the auxotrophic factor of the present invention may be administered rectally and/or vaginally. Compositions for rectal or vaginal administration are typically suppositories which can be prepared by mixing compositions with suitable non-irritating excipients such as cocoa butter, polyethylene glycol, or a suppository wax which are solid at ambient temperature but liquid at body temperature and therefore melt in the rectum or vaginal cavity and release the active ingredient.

### Oral Administration

In some embodiments, pharmaceutical compositions including the regulatable cell line of the auxotrophic factor of the present invention may be administered orally. Solid dosage forms for oral administration include capsules, tablets, pills, powders, and granules. In such solid dosage forms, an active ingredient is mixed with at least one inert, pharmaceutically acceptable excipient such as sodium citrate or dicalcium phosphate and/or fillers or extenders (e.g. starches, lactose, sucrose, glucose, mannitol, and silicic acid), binders (e.g. carboxymethylcellulose, alginates, gelatin, polyvinylpyrrolidinone, sucrose, and acacia), humectants (e.g. glycerol), disintegrating agents (e.g. agar, calcium carbonate, potato or tapioca starch, alginic acid, certain silicates, and sodium carbonate), solution retarding agents (e.g. paraffin), absorption accelerators (e.g. quaternary ammonium compounds), wetting agents (e.g. cetyl alcohol and glycerol monostearate), absorbents (e.g. kaolin and bentonite clay), and lubricants (e.g. talc, calcium stearate, magnesium stearate, solid polyethylene glycols, sodium lauryl sulfate), and mixtures thereof. In the case of capsules, tablets and pills, the dosage form may include buffering agents.

### Depot Administration

As described herein, in some embodiments, pharmaceutical compositions including the regulatable cell line of the present invention are formulated in depots for extended release. Generally, specific organs or tissues (“target tissues”) are targeted for administration. In some embodiments, localized release is effected via utilization of a biocompatible device. For example, the biocompatible device may restrict diffusion of the cell line in the subject.

In some aspects of the invention, pharmaceutical compositions including the regulatable cell line of the present invention are spatially retained within or proximal to target tissues. Provided are methods of providing pharmaceutical compositions including the regulatable cell line or the auxotrophic factor, to target tissues of mammalian subjects by contacting target tissues (which include one or more target cells) with pharmaceutical compositions including the regulatable cell line or the auxotrophic factor, under conditions such that they are substantially retained in target tissues, meaning that at least 10, 20, 30, 40, 50, 60, 70, 80, 85, 90, 95, 96, 97, 98, 99, 99.9, 99.99, or greater than 99.99% of the composition is retained in the target tissues. For example, at least 1%, 5%, 10%, 20%, 30%, 40%, 50%, 60%, 70%, 80%, 85%, 90%, 95%, 96%, 97%, 98%, 99%, 99.9%, 99.99% or greater than 99.99% of pharmaceutical compositions including the regulatable cell line or the auxotrophic factor administered to subjects are present at a period of time following administration.

Certain aspects of the invention are directed to methods of providing pharmaceutical compositions including the regulatable cell line or the auxotrophic factor of the present invention to target tissues of mammalian subjects, by contacting target tissues with pharmaceutical compositions including the regulatable cell line under conditions such that they are substantially retained in such target tissues. Pharmaceutical compositions including the regulatable cell line include enough active ingredient such that the effect of interest is produced in at least one target cell. In some embodiments, pharmaceutical compositions including the regulatable cell line generally include one or more cell penetration agents, although “naked” formulations (such as without cell penetration agents or other agents) are also contemplated, with or without pharmaceutically acceptable carriers.

### Pulmonary Administration

In some embodiments, pharmaceutical compositions including the regulatable cell line or the auxotrophic factor of the present invention may be prepared, packaged, and/or sold in formulations suitable for pulmonary administration. In some embodiments, such administration is via the buccal cavity. In some embodiments, formulations may include dry particles including active ingredients. In such embodiments, dry particles may have a diameter in the range from about 0.5 nm to about 7 nm or from about 1 nm to about 6 nm. In some embodiments, formulations may be in the form of dry powders for administration using devices including dry powder reservoirs to which streams of propellant may be directed to disperse such powder. In some embodiments, self-propelling solvent/powder dispensing containers may be used. In such embodiments, active ingredients may be dissolved and/or suspended in low-boiling propellant in sealed containers. Such powders may include particles such that at least 98% of the particles by weight have diameters greater than 0.5 nm and at least 95% of the particles by number have diameters less than 7 nm. Alternatively, at least 95% of the particles by weight have a diameter greater than 1 nm and at least 90% of the particles by number have a diameter less than 6 nm. Dry powder compositions may include a solid fine powder diluent such as sugar and are conveniently provided in a unit dose form.

Low boiling propellants generally include liquid propellants having a boiling point of below 18° C. at atmospheric pressure. Generally, propellants may constitute 50% to 99.9% (w/w) of the composition, and active ingredient may constitute 0.1% to 20% (w/w) of the composition. Propellants may further include additional ingredients such as liquid non-ionic and/or solid anionic surfactant and/or solid diluent (which may have particle sizes of the same order as particles including active ingredients).

Pharmaceutical compositions formulated for pulmonary delivery may provide active ingredients in the form of droplets of solution and/or suspension. Such formulations may be prepared, packaged, and/or sold as aqueous and/or dilute alcoholic solutions and/or suspensions, optionally sterile, including active ingredients, and may conveniently be administered using any nebulization and/or atomization device. Such formulations may further include one or more additional ingredients including, but not limited to, a flavoring agent such as saccharin sodium, a volatile oil, a buffering agent, a surface active agent, and/or a preservative such as methylhydroxybenzoate. Droplets provided by this route of administration may have an average diameter in the range from about 0.1 nm to about 200 nm.

### Intranasal, Nasal, and Buccal Administration

In some embodiments, pharmaceutical compositions including the regulatable cell line or the auxotrophic factor of the present invention may be administered nasally and/or intranasally. In some embodiments, formulations described herein useful for pulmonary delivery may also be useful for intranasal delivery. In some embodiments, formulations for intranasal administration include a coarse powder including the active ingredient and having an average particle from about 0.2 μm to 500 μm. Such formulations are administered in the manner in which snuff is taken, i.e. by rapid inhalation through the nasal passage from a container of the powder held close to the nose.

Formulations suitable for nasal administration may, for example, include from about as little as 0.1% (w/w) and as much as 100% (w/w) of active ingredient, and may include one or more of the additional ingredients described herein. A pharmaceutical composition may be prepared, packaged, and/or sold in a formulation suitable for buccal administration. Such formulations may, for example, be in the form of tablets and/or lozenges made using conventional methods, and may, for example, 0.1% to 20% (w/w) active ingredient, the balance including an orally dissolvable and/or degradable composition and, optionally, one or more of the additional ingredients described herein. Alternately, formulations suitable for buccal administration may include powders and/or an aerosolized and/or atomized solutions and/or suspensions including active ingredients. Such powdered, aerosolized, and/or aerosolized formulations, when dispersed, may include average particle and/or droplet sizes in the range of from about 0.1 nm to about 200 nm, and may further include one or more of any additional ingredients described herein.

### Ophthalmic or Otic Administration

In some embodiments, pharmaceutical compositions including the regulatable cell line or the auxotrophic factor of the present invention may be prepared, packaged, and/or sold in formulations suitable for ophthalmic and/or otic administration. Such formulations may, for example, be in the form of eye and/or ear drops including, for example, a 0.1/1.0% (w/w) solution and/or suspension of the active ingredient in aqueous and/or oily liquid excipients. Such drops may further include buffering agents, salts, and/or one or more other of any additional ingredients described herein. Other ophthalmically-administrable formulations which are useful include those which include active ingredients in microcrystalline form and/or in liposomal preparations. Subretinal inserts may also be used as forms of administration.

### Delivery to Cells

The present disclosure provides a method of treating a disease, a disorder, or a condition in a subject with a regulatable cell line, the method including: generating a cell line which is auxotrophic for a nutrient, an enzyme, an altered pH, an altered temperature, an altered concentration of a moiety, and/or a niche environment such that the nutrient, enzyme, altered pH, altered temperature, concentration of a moiety, or niche environment is not present in the subject; contacting the subject with the resulting auxotrophic cell line of the generating step; contacting the subject with an auxotrophic factor which is selected from a nutrient, an enzyme, a moiety that alters pH and/or temperature, an increase in concentration of a moiety, and a cellular niche environment in the subject which was not previously present in the subject, such that the auxotrophic factor activates the auxotrophic system or element resulting in the growth of the cell line and/or the expression of one or more therapeutic entities for the subject. The auxotrophic factor may be delivered to cells of these regulatable cell lines by any of the administration techniques mentioned herein.

### Delivery to Subjects

The present disclosure additionally provides a method of delivering to a subject, including a mammalian subject, any of the above-described regulatable cell lines or auxotrophic factors including administering to the subject the regulatable cell lines or auxotrophic factors, or administering to the subject a formulation including the regulatable cell lines or auxotrophic factors, or administering to the subject any of the described compositions, including pharmaceutical compositions.

### Dose and Regimen

The present invention provides methods of administering regulatable cell lines or auxotrophic factors in accordance with the invention to a subject in need thereof. The pharmaceutical compositions including the regulatable cell line or the auxotrophic factor and compositions of the present invention may be administered to a subject using any amount and any route of administration effective for preventing, treating, managing, or diagnosing diseases, disorders and/or conditions. The exact amount required will vary from subject to subject, depending on the species, age, and general condition of the subject, the severity of the disease, the particular composition, its mode of administration, its mode of activity, and the like. The subject may be a human, a mammal, or an animal. Compositions in accordance with the invention are typically formulated in unit dosage form for ease of administration and uniformity of dosage. It will be understood, however, that the total daily usage of the compositions of the present invention may be decided by the attending physician within the scope of sound medical judgment. The specific therapeutically effective, prophylactically effective, or appropriate diagnostic dose level for any particular individual will depend upon a variety of factors including the disorder being treated and the severity of the disorder; the activity of the specific payload employed; the specific composition employed; the age, body weight, general health, sex and diet of the patient; the time of administration, route of administration, and rate of excretion of the specific regulatable cell line or auxotrophic factor; the duration of the treatment; drugs used in combination or coincidental with the specific regulatable cell line or auxotrophic factor particle employed, and like factors well known in the medical arts.

In certain embodiments, regulatable cell line or the auxotrophic factor pharmaceutical compositions in accordance with the present invention may be administered at dosage levels sufficient to deliver from about 0.0001 mg/kg to about 100 mg/kg, from about 0.001 mg/kg to about 0.05 mg/kg, from about 0.005 mg/kg to about 0.05 mg/kg, from about 0.001 mg/kg to about 0.005 mg/kg, from about 0.05 mg/kg to about 0.5 mg/kg, from about 0.01 mg/kg to about 50 mg/kg, from about 0.1 mg/kg to about 40 mg/kg, from about 0.5 mg/kg to about 30 mg/kg, from about 0.01 mg/kg to about 10 mg/kg, from about 0.1 mg/kg to about 10 mg/kg, or from about 1 mg/kg to about 25 mg/kg, of subject body weight per day, one or more times a day, to obtain the desired therapeutic, diagnostic, or prophylactic, effect.

In certain embodiments, regulatable cell line or auxotrophic factor pharmaceutical compositions in accordance with the present disclosure may be administered at about 10 to about 600 μl/site, 50 to about 500 μl/site, 100 to about 400 μl/site, 120 to about 300 μl/site, 140 to about 200 μl/site, about 160 μl/site. As non-limiting examples, the regulatable cell line or auxotrophic factor may be administered at 50 μl/site and/or 150 μl/site.

The desired dosage of the regulatable cell line or auxotrophic factor of the present invention may be delivered only once, three times a day, two times a day, once a day, every other day, every third day, every week, every two weeks, every three weeks, or every four weeks. In certain embodiments, the desired dosage may be delivered using multiple administrations (e.g., two, three, four, five, six, seven, eight, nine, ten, eleven, twelve, thirteen, fourteen, or more administrations). When multiple administrations are employed, split dosing regimens such as those described herein may be used. As used herein, a “split dose” is the division of “single unit dose” or total daily dose into two or more doses, e.g., two or more administrations of the “single unit dose”. As used herein, a “single unit dose” is a dose of any therapeutic administered in one dose/at one time/single route/single point of contact, i.e., single administration event.

The desired dosage of the regulatable cell lines of the present invention may be administered as a “pulse dose” or as a “continuous flow”. As used herein, a “pulse dose” is a series of single unit doses of any therapeutic administered with a set frequency over a period of time. As used herein, a “continuous flow” is a dose of therapeutic administered continuously for a period of time in a single route/single point of contact, i.e., continuous administration event. A total daily dose, an amount given or prescribed in 24-hour period, may be administered by any of these methods, or as a combination of these methods, or by any other methods suitable for a pharmaceutical administration.

In one embodiment, delivery of the regulatable cell line or auxotrophic factor of the present invention to a subject provides neutralizing activity to a subject. The neutralizing activity can be for at least 1 month, 2 months, 3 months, 4 months, 5 months, 6 months, 7 months, 8 months, 9 months, 10 months, 11 months, 1 year, 13 months, 14 months, 15 months, 16 months, 17 months, 18 months, 19 months, 20 months, 20 months, 21 months, 22 months, 23 months, 2 years, 3 years, 4 years, 5 years, 6 years, 7 years, 8 years, 9 years, 10 years or more than 10 years.

### Combinations

The regulatable cell lines may be used in combination with one or more other therapeutic, prophylactic, research or diagnostic agents. By “in combination with”, it is not intended to imply that the agents must be administered at the same time and/or formulated for delivery together, although these methods of delivery are within the scope of the present invention. Compositions can be administered concurrently with, prior to, or subsequent to, one or more other desired therapeutics or medical procedures. In general, each agent will be administered at a dose and/or on a time schedule determined for that agent. In some embodiments, the present disclosure encompasses the delivery of pharmaceutical, prophylactic, research, or diagnostic compositions in combination with agents that may improve their bioavailability, reduce and/or modify their metabolism, inhibit their excretion, and/or modify their distribution within the body.

### Bioavailability

The regulatable cell line or auxotrophic factor, when formulated into a composition as described herein, can exhibit an increase in bioavailability as compared to a composition not formulated as described herein. As used herein, the term “bioavailability” refers to the systemic availability of a given amount of the regulatable cell line or auxotrophic factor administered to a subject. Bioavailability can be assessed by measuring the area under the curve (AUC) or the maximum serum or plasma concentration (Cₘₐₓ) of the composition following administration. AUC is a determination of the area under the curve plotting the serum or plasma concentration of a compound (e.g., regulatable cell line or auxotrophic factor) along the ordinate (Y-axis) against time along the abscissa (X-axis). Generally, the AUC for a particular compound can be calculated using methods known to those of ordinary skill in the art and as described in G. S. Banker, Modern Pharmaceutics, Drugs and the Pharmaceutical Sciences, v. 72, Marcel Dekker, New York, Inc., 1996, the contents of which are herein incorporated by reference in its entirety.

The Cₘₐₓ value is the maximum concentration of the regulatable cell line or auxotrophic factor achieved in the serum or plasma of a mammal following administration of the regulatable cell line or auxotrophic factor to the mammal. The Cₘₐₓ value of can be measured using methods known to those of ordinary skill in the art. The phrases “increasing bioavailability” or “improving the pharmacokinetics,” as used herein mean that the systemic availability of a regulatable cell line or auxotrophic factor, measured as AUC, Cₘₐₓ, or Cₘᵢₙ in a mammal is greater, when administered as described herein, than prior to administration. In some embodiments, the bioavailability can increase by at least about 2%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or about 100%.

For example, the pharmaceutical composition is administered as a biocompatible device that restricts diffusion of the regulatable cell line in the subject to increase bioavailability in the area targeted for treatment. Bioavailability may also be altered by localized release of the auxotrophic factor, such as a nutrient or enzyme.

### Therapeutic Window

As used herein “therapeutic window” refers to the range of plasma concentrations, or the range of levels of therapeutically active substance at the site of action, with a high probability of eliciting a therapeutic effect. In some embodiments, the therapeutic window of the regulatable cell line or auxotrophic factor as described herein can increase by at least about 2%, at least about 5%, at least about 10%, at least about 15%, at least about 20.1%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or about 100%.

### Volume of Distribution

As used herein, the term “volume of distribution” refers to the fluid volume that would be required to contain the total amount of the drug in the body at the same concentration as in the blood or plasma: Vdist equals the amount of drug in the body/concentration of drug in blood or plasma. For example, for a 10 mg dose and a plasma concentration of 10 mg/L, the volume of distribution would be 1 liter. The volume of distribution reflects the extent to which the drug is present in the extravascular tissue. A large volume of distribution reflects the tendency of a compound to bind to the tissue components compared with plasma protein binding. In a clinical setting, Vdist can be used to determine a loading dose to achieve a steady state concentration. In some embodiments, the volume of distribution of the regulatable cell line or auxotrophic factor as described herein can decrease at least about 2%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%.

### Biological Effect

In one embodiment, the biological effect of the regulatable cell line or auxotrophic factor delivered to the animals may be categorized by analyzing the payload expression in the animals.

### VI. Methods of Creating the Regulatable Cell Lines of the Invention

As described above, methods for gene editing are well known in the art. Thus, the invention provides a method of creating a regulatable human cell line comprising the steps of: (a) obtaining a pool of cells, (b) using a nuclease to introduce an addition, deletion or substitution of one or more nucleotides in a target gene, thereby knocking out or downregulating expression of that gene, and (c) screening for auxotrophy.

In one embodiment, the target gene is selected from those disclosed in Table 1. In one embodiment the target gene is selected from uridine monophosphate synthetase (UMPS) (creating a cell line auxotrophic for uracil) and holocarboxylase synthetase (creating a cell line auxotrophic for biotin). The screening step may be carried out by culturing the cells with or without one of the auxotrophic factors disclosed in Table 1. In one embodiment, the auxotrophic factor is selected from biotin, alanine, aspartate, asparagine, glutamate, serine, uracil and cholesterol. In one embodiment, the auxotrophic factor is selected from biotin and uracil.

In one embodiment the cells are selected from lymphocytes (such as T cells), induced pluripotent stem (iPS) cells, embryonic stem cells, somatic stem cells, haematopoetic stem cells and peripheral blood mononuclear cells (PBMCs).

In one embodiment, the nuclease is a CRISPR/Cas nuclease, such as those described above, for example CRISPR/Cas9.

### VII. Definitions

The term “about” in relation to a numerical value x means, for example, x±10%.

The term “active ingredient” as used herein, refers to the regulatable cell line including an engineered genome encoding an auxotrophic response system or element as described herein. Alternatively, the term refers to the auxotrophic factor as described herein.

The term “altered concentration” as used herein, refers to an increase in concentration of an auxotrophic factor compared to the concentration of the auxotrophic factor in the subject prior to administration of the pharmaceutical compositions described herein.

The term “altered pH” as used herein, refers to a change in pH induced in a subject compared to the pH in the subject prior to administration of the pharmaceutical composition described herein.

The term “altered temperature” as used herein refers to a change in temperature induced in a subject compared to the temperature in the subject prior to administration of the pharmaceutical composition as described herein.

The term “auxotrophy” as used herein, refers to is a condition of a cell that causes an inability of the cell to produce a compound required for proliferation.

The term “auxotrophic cell line” as used herein, refers to a cell line including a genome that is genetically engineered to include an auxotrophic response system or element, and that is capable of growth only in the presence of the auxotrophic factor.

The term, “auxotrophic factor” as used herein, refers to a nutrient, an enzyme, a moiety that alters pH, a moiety that alters temperature, or a cellular niche environment in a subject which was not previously present in the subject, that activates the auxotrophic response system or element resulting in the growth of the cell line and/or the expression of one or more therapeutic entities.

The term “auxotrophic response system or element” as used herein, refers to a portion of a genome of the regulatable cell lines described herein that is responsive to the presence of an auxotrophic factor.

The term “bioavailability” as used herein, refers to systemic availability of a given amount of the regulatable cell line or auxotrophic factor administered to a subject.

The term “cell line” as used herein, refers to a population of cells descended from the same cell, such that each cell of the population includes a similar genetic make-up.

The term “Cas9” as used herein, refers to CRISPR-associated protein 9, which is an endonuclease for use in genome editing.

The term “CRISPR” as used herein, refers to clustered regularly interspaced short palindromic repeats of DNA that deploy an enzyme that cuts the RNA nucleotides of an invading cell.

The term “CRISPR/Cas9 platform” as used herein, refers to a genetic engineering tool that includes a guide RNA (gRNA) sequence with a binding site for Cas9 and a targeting sequence specific for the area to be modified. The Cas9 binds the gRNA to form a ribonucleoprotein that binds and cleaves the target area.

The term “comprising” means “including” as well as “consisting” e.g. a composition “comprising” X may consist exclusively of X or may include something additional e.g. X+Y.

The term “continuous flow” as used herein, refers to a dose of therapeutic administered continuously for a period of time in a single route/single point of contact, i.e., continuous administration event.

The term “engineered genome” as used herein, refers to the genome of a cell line that has been altered using molecular biology techniques that are known in the art.

The term “inactive ingredient” as used herein, refers to one or more agents that do not contribute to the activity of the active ingredient of the pharmaceutical composition included in formulations.

The term “gene knockout cell lines” as used herein, refers to a cell line with a targeted disruption of a gene resulting in complete loss of function that has been achieved by a molecular biology technology known in the art.

The term “pharmaceutical composition” as used herein, refers to a composition including at least one active ingredient and optionally one or more pharmaceutically acceptable excipients.

The term “pharmaceutically acceptable salt” as used herein, refers to derivatives of the disclosed compounds such that the parent compound is modified by converting an existing acid or base moiety to its salt form (e.g., by reacting the free base group with a suitable organic acid).

The term “pulse dose” as used herein, refers to a series of single unit doses of any therapeutic administered with a set frequency over a period of time.

The term “niche environment” as used herein, refers to a habitat for the regulatable cell line described herein that supplies the factors necessary for proliferation or production of a therapeutic entity.

The term “nutrient” as used herein, refers to a substance that may be a chemical element or compound utilized in metabolism and anabolism in an organism.

The term “regenerative” as used herein, refers to renewal or restoration of an organ or system of the subject.

The term “regulatable cell line” as used herein, refers to a cell line that is genetically engineered to include an auxotrophic response system or element.

The term “therapeutic product” or “therapeutic entity” as used herein, refers to a product encoded by the regulatable cell line that treats and/or alleviates symptoms of the disease, disorder, or condition of the subject.

The term “therapeutic window” as used herein, refers to the range of plasma concentrations, or the range of levels of therapeutically active substance at the site of action, with a high probability of eliciting a therapeutic effect.

The term “translational switch” as used herein, refers to a portion of an auxotrophic response system or element that upon exposure to the auxotrophic factor, increases translation of at least one gene transcript.

The term “unit dose” as used herein, refers to a discrete amount of the pharmaceutical composition including a predetermined amount of the active ingredient.

The term “volume of distribution” as used herein, refers to a fluid volume that would be required to contain the total amount of the drug in the body at the same concentration as in the blood or plasma: V_{dist} equals the amount of drug in the body/concentration of drug in blood or plasma.

The details of one or more embodiments of the invention are set forth in the accompanying description below. Although any materials and methods similar or equivalent to those described herein can be used in the practice or testing of the present invention, the preferred materials and methods are now described. Other features, objects and advantages of the invention will be apparent from the description. In the description, the singular forms also include the plural unless the context clearly dictates otherwise. Unless defined otherwise, all technical and scientific terms used herein have the same meaning as commonly understood by one of ordinary skill in the art to which this invention belongs. In the case of conflict, the present description will control.

The present invention is further illustrated by the following non-limiting examples.

### BRIEF DESCRIPTION OF THE FIGURES

FIG. 1 shows that serum is required for optimal recovery post-electroporation. (A) Schematic of assay used to determine optimal electroporation recovery conditions. Following electroporation, cells were supplied with/without serum, 5-fluoroorotic acid (5-FOA), or an exogenous uracil source (uridine). (B) Cell counts by Cytoflex (Beckman Coulter) flow cytometer after 4 days of recovery post electroporation in indicated media conditions. The figure shows cells administered serum, mock edited cells treated with/without 5-FOA with no serum, and uridine monophosphate synthetase (UMPS) knockout cells treated with/without 5-FOA without serum.

FIG. 2 shows that maintenance and growth of UMPS InDel containing cells requires an exogenous uracil source. (A) Schematic of the procedure used to assay for growth of UMPS or mock edited T cells following electroporation and recovery. (B) TIDE analysis of UMPS InDels in indicated culture conditions. TIDE analysis was performed on sanger sequencing of UMPS locus with oligonucleotides UMPS-O-1 and UMPS-O-2. Performed on day 8. (C) Percentage of alleles analysed by TIDE containing frameshift InDels. Performed on day 8. (D) Predicted absolute numbers of cells at day 8 containing alleles identified by TIDE. (E) Time course of cell counts with/without UMP. (F) Time course of cell counts with/without uridine.

FIG. 3 shows 5-FOA is less toxic in UMPS targeted cell lines. (A) Schematic of 5-FOA selection procedure. (B-C) Cell counts after 4 days of 5-FOA selection in indicated culture conditions.

FIG. 4 shows that 5-FOA selected, UMPS targeted cell lines exhibit optimum growth only in the presence of an exogenous uracil source. (A) Schematic of protocol for the demonstration of uracil auxotrophy. Cell cultures were split following 4-day selection in 5-FOA into test media and grown for 4 further days before cell counting. (B-D) Cell counts of 5-FOA selected cells in exogenous uracil (UMP or uridine) containing or deficient media.

### EXAMPLES

### Example 1: General T Cell Culture Methods

Buffy coats were obtained and T cells isolated through Ficoll density gradient centrifugation followed by magnetic enrichment using the Pan T cell isolation kit (Miltenyi).

Cells were cryopreserved in Bambanker medium. After thawing cells were cultured at 37° C., 5% CO₂ in X-Vivo 15 (Lonza) supplemented with or without 5% human serum (Sigma-Aldrich) and 100 IU/ml human recombinant IL-2 (Peprotech) and 10 ng/ml human recombinant IL-7 (BD Biosciences). UMP or Uridine were added at 250 ug/ml. 5-FOA was added at 100 ug/ml to 1 mg/ml. During culture, medium was refreshed every 2 days.

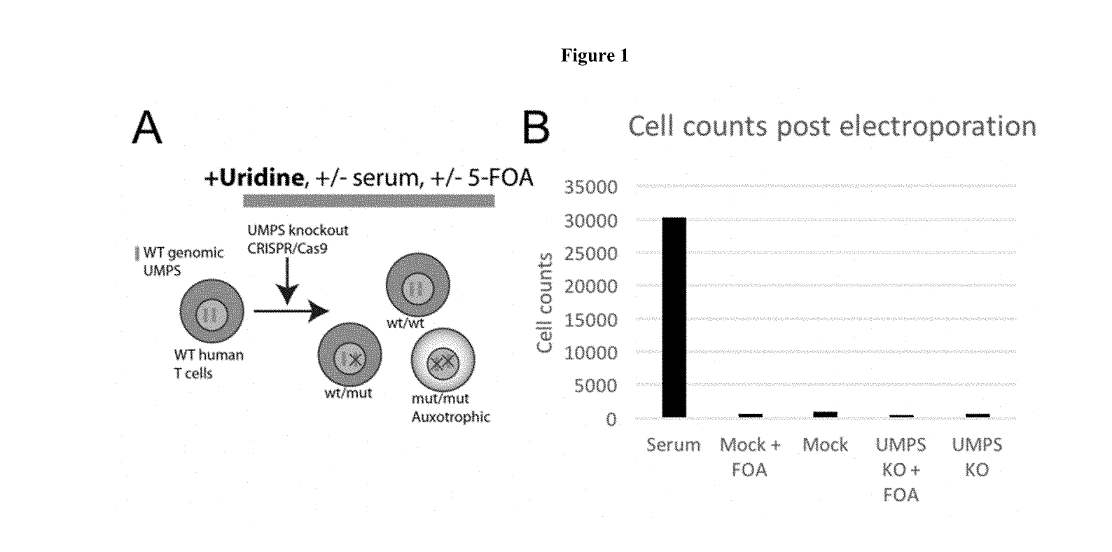

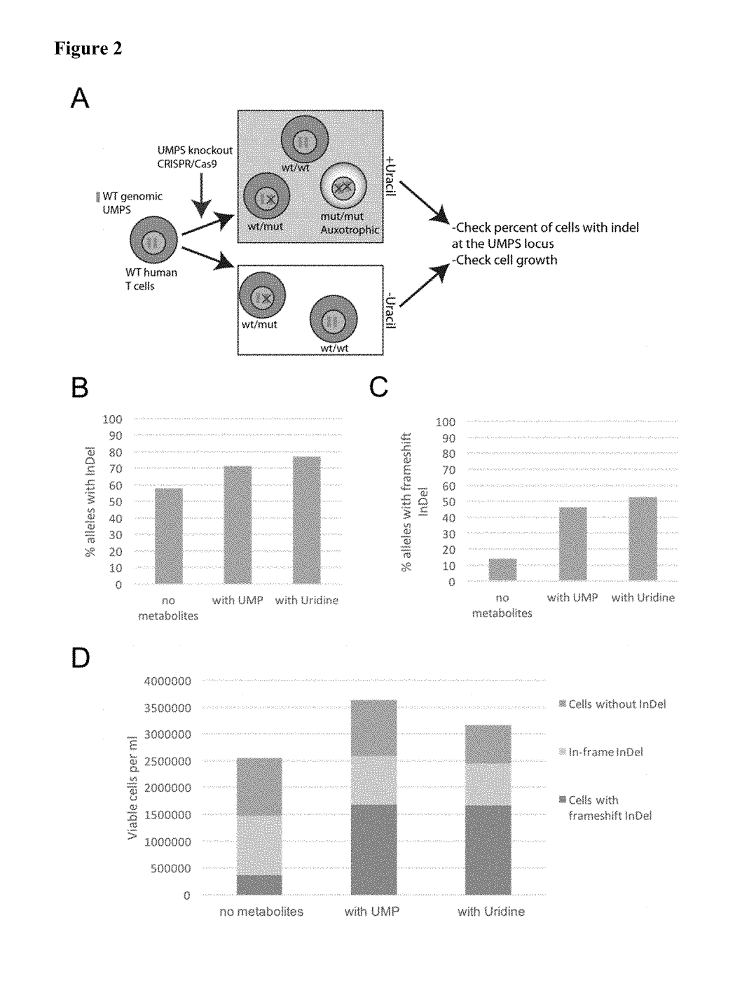

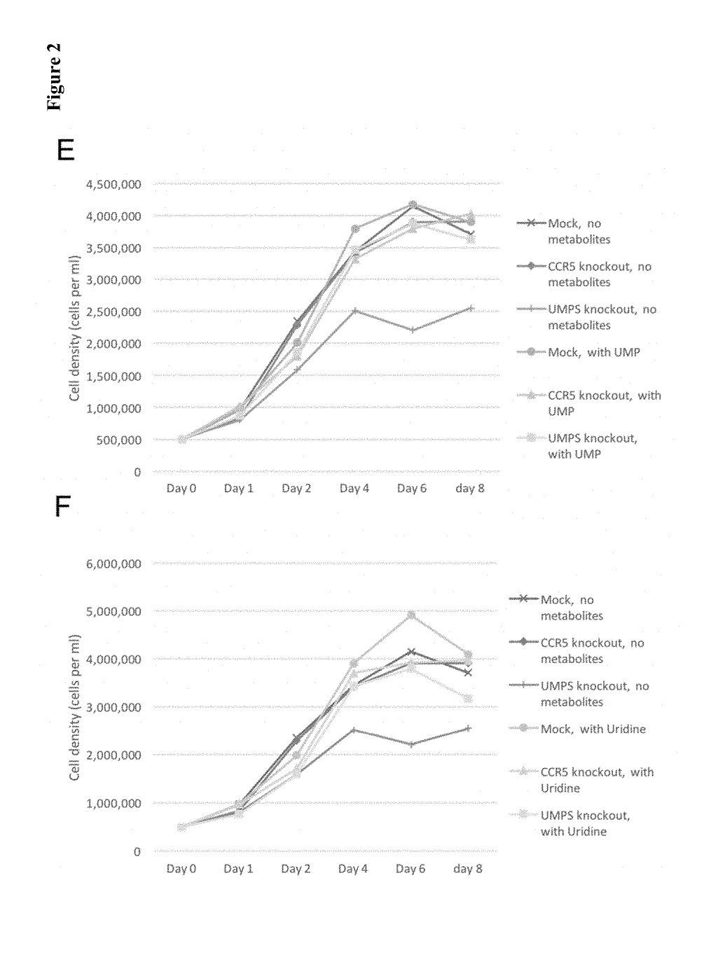

T cells were activated using immobilized Anti-CD3 (clone OKT3, Tonbo Biosciences) and soluble anti-CD28 (clone CD28.2, Tonbo Biosciences) for 3 days before electroporation. For the experiments in FIGS. 1, 3 and 4, 1.4 million activated T cells were electroporated. For the experiments in FIG. 2, 30,000 activated T cells were electroporated per condition. These cells were resuspended in electroporation solution, mixed with the pre-complexed RNP and electroporated using a 4D-Nucleofector (Lonza) using program EO-115. The RNP consisted of Cas9 protein (Alt-R® S. pyogenes, IDT) at 300 ug/ml and sgRNA using a sgRNA:Cas9 molar ratio of 2.5.

Genomic DNA was harvested using QuickExtract (Epicentre). Cells were counted on an automated cell counter using Trypan blue staining or on a Cytoflex (Beckman Coulter) flow cytometer using CountBright beads (ThermoFisher) as a reference. Data was analyzed using Excel (Microsoft) and FlowJo (Tree Star).

Sanger sequencing of the UMPS locus was performed using UMPS-O-1 and UMPS-O-2, with the region amplified using Phusion Hotstart Flex Mastermix (NEB). Sanger sequencing traces were analysed by TIDE (Brinkman et al, 2014) to identify insertions and deletions (InDels) after editing.

gRNA Sequences (Including PAMs)

UMPS-7
(SEQ ID NO: 1)
GCC CCG CAG AUC GAU GUA GAG UUU UAG AGC UAG AAA
UAG CAA GUU AAA AUA AGG CUA GUC CGU UAU CAA CUU
GAA AAA GUG GCA CCG AGU CGG UGC UUU U

Sequencing Oligos for UMPS Locus TIDE Analysis:

UMPS-O-1:
(SEQ ID NO: 2)
CCCGGGGAAACCCACGGGTGC
UMPS-O-2:
(SEQ ID NO: 3)
AGGGTCGGTCTGCCTGCTTGGCT

### Example 2: UMPS Editing by Cas9-sgRNA Electroporation in Human T Cells

T Cells were thawed and cultured, followed by activation and subsequent electroporation with Cas9-UMPS-7 sgRNA RNP as described above. Following electroporation, cells were allowed to recover in medium with or without serum, 5-FOA or an exogenous uracil source (FIG. 1A). Cell survival following electroporation was markedly increased when serum was included in the media (FIG. 1B).

### Example 3: Optimal Growth of a Mixed UMPS Edited Population and Maintenance of UMPS Mutations Requires an Exogenous Uracil Source

T Cells were electroporated and edited as in Example 2, and allowed to recover for a 2 day period in medium+serum+uridine+UMP. On day 0, cells were shifted to UMP, Uridine or −uracil source media. This experiment does not feature a selection step and thus the resulting population of cells is a heterogeneous mix of wildtype (WT), heterozygous mutant and homozygous mutant cells. The growth of homozygous UMPS mutant cells should be dependent on an exogenous uracil source—as these should be auxotrophic (FIG. 2A). When UMPS is targeted, InDels are generated in ˜55-75% of cells (as assayed by TIDE (Brinkman et al, 2014, Nucleic Acids Res. 42(22):e168)) (FIG. 2B). When the media is uracil deficient, the frameshift generating InDel frequency in the population is reduced (FIG. 2C). This is consistent with the model that any homozygous auxotrophic UMPS mutant cells would be outcompeted in the population by non-auxotrophic heterozygous mutants and WT cells still present after editing—resulting in a reduced predicted number of frameshift InDel containing cells (FIG. 2D). The optimal growth of the heterogeneous UMPS edited population is also dependent on the presence of an exogenous source of uracil (FIG. 2E-F). UMP and uridine rescue the growth of UMPS edited cells to the same level as mock edited and CCR5 targeted controls (a control editing locus). This decreased cell growth rate in the absence of exogenous uracil is dependent on UMPS editing, and is not seen in any of the control cells, indicating that edited UMPS causes human T cells to be specifically dependent on uracil supplementation for optimal cell growth.

It is worth reiterating the UMPS edited population contains unedited or heterozygous cells that are not expected to be auxotrophic, and thus complete lack of growth of UMPS edited cells in uracil deficient media is not expected.

### Example 4: 5-FOA Treatment Selects for UMPS Targeted Cells

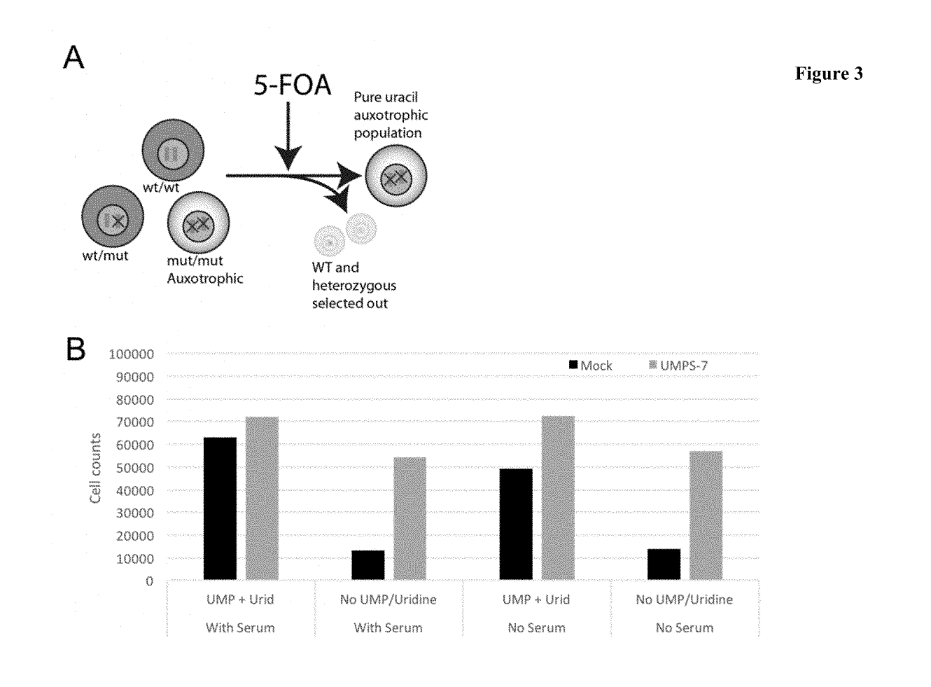

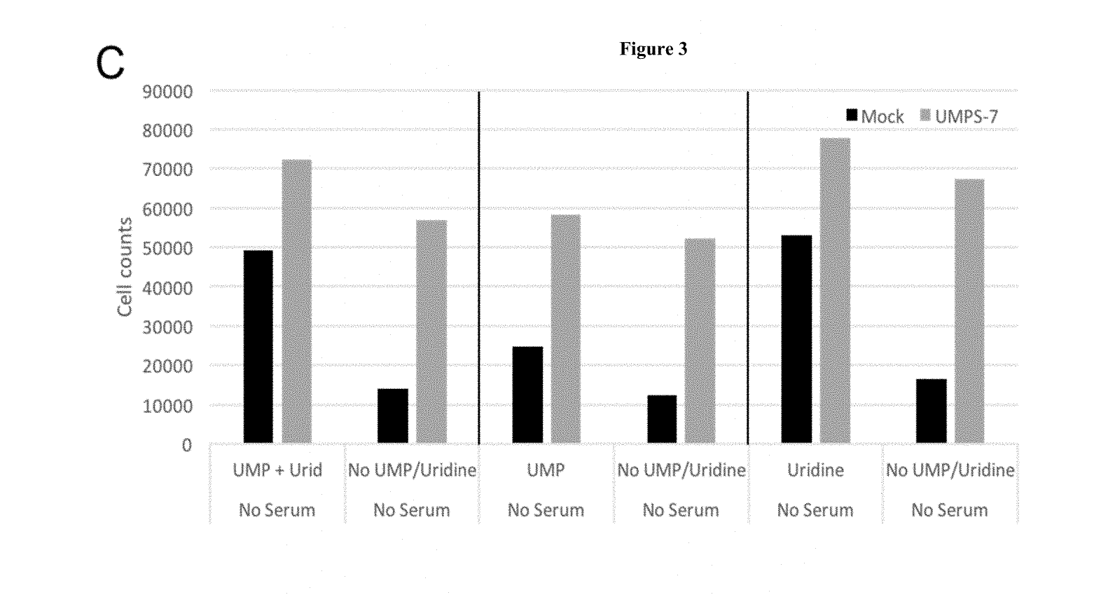

5-FOA selects for uracil auxotrophic cells in other organisms (e.g. Boeke et al. 1984, Mol. Gen. Genet. 197(2):345-6). To investigate the potential utility of 5-FOA for the selection of uracil auxotrophs in human cells, the UMPS gene was targeted in human T cells by Cas9-gRNA complex electroporation followed by recovery (as in Example 2) followed by an assay of resistance to 5-FOA treatment (FIG. 3A). Cells were grown in 5-FOA and a variety of combinations of serum and uracil sources for 4 days before cell counting was performed. Serum, while important for the recovery of cells post electroporation, had no effect on the viability of cells in 5-FOA (FIG. 3B). Uridine and UMP improved the survival of both mock treated and UMPS targeted cells in 5-FOA. This is likely through a competition based mechanism (uridine can reverse 5-fluorouracil toxicity in humans (van Groeningen et al. 1992, Semin. Oncol. 19(2 Suppl 3):148-54)) (FIGS. 3B and C). In all cases, UMPS targeted cells exhibited increased survival vs. mock targeted cells. This data indicates that 5-FOA can be used for the selection of uracil auxotrophic cells in human cell culture.

### Example 5: 5-FOA Selected UMPS Targeted Cells Exhibit Uracil Auxotrophy

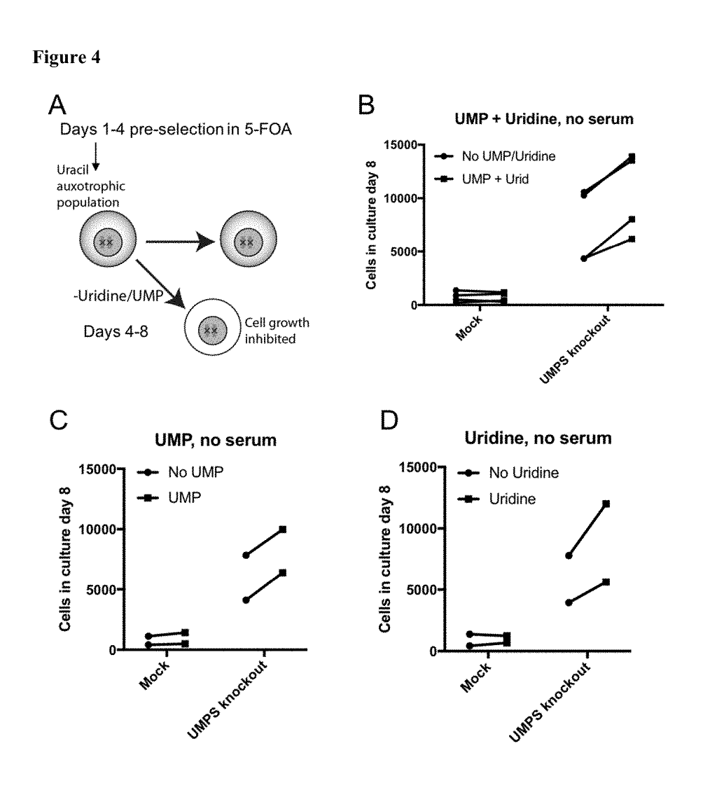

To assay whether or not the cells selected for by 5-FOA treatment are uracil auxotrophs, mock or UMPS targeted T cells were exposed to 5-FOA as in Example 4. Following 4 days of 5-FOA selection, the population of cells was split into a uracil containing media (either UMP, uridine or both) and a uracil deficient media. A growth assay was subsequently performed by cell counting after following 4 days incubation in test media (day 8) (FIG. 4A). In all cases, cell growth in the mock targeted cell cultures was negligible and independent of uracil source supplementation—indicating successful killing of non-UMPS targeted cells during the 5-FOA selection step (FIG. 4B-D). In the UMPS targeted population, in all conditions cell growth was stimulated by the addition of uracil and poor cell growth was observed in its absence (FIG. 4B-D).

Taken together, the results of Examples 1-5 indicate that editing of the UMPS locus by Cas9 in human T cells generates cells that are dependent on an exogenous uracil source for optimal cell growth. These results demonstrate that engineered human auxotrophy can be used as a mechanism for controlling the proliferation of T cells or some other cell therapy. In addition, 5-FOA selection of UMPS edited cells provides a useful mechanism for selection of a true auxotrophic population of T cells.

### Example 6: Culturing of Stem Cells

The regulatable cell lines that are the subject matter of the invention herein may include stem cells that were maintained and differentiated using the techniques below as shown in U.S. Pat. No. 8,945,862, which is hereby incorporated by reference in its entirety.

Undifferentiated hES cells (H9 line from WiCell®, passages 35 to 45) are grown on an inactivated mouse embryonic fibroblast (MEF) feeder layer (Stem Cells, 2007. 25(2): p. 392-401). Briefly, the cell is maintained at an undifferentiated stage on irradiated low-passage MEF feeder layers on 0.1% gelatin-coated plates. The medium is changed daily. The medium consists of Dulbecco's modified Eagle's medium (DMEM)/F-12, 20% knockout serum replacement, 0.1 mM nonessential amino acids, 2 mM L-glutamine, 0.1 mM β-mercaptoethanol, and 4 ng/ml rhFGF-2 (R&D Systems Inc., Minneapolis). The undifferentiated hES cells are treated by 1 mg/ml collagenase type IV in DMEM/F12 and scraped mechanically on the day of passage. Prior to differentiation, hES cell are seeded onto Matrigel®-coated plates in conditioned medium (CM) prepared from MEF as follows (Nat Biotechnol, 2001. 19(10): p. 971-4). MEF cells are harvested and irradiated with 50 Gy, and are cultured with hES medium without bFGF. CM is collected daily and supplemented with an additional 4 ng/ml of bFGF before feeding hES cells.

### Example 7: In Vitro Differentiation of hESC-ECs

To induce hES cell differentiation, undifferentiated hES cells are cultured in differentiation medium containing Iscove's modified Dulbecco's medium (IMDM) and 15% defined fetal bovine serum (FBS) (Hyclone, Logan, Utah), 0.1 mM nonessential amino acids, 2 mM L-glutamine, 450 μM monothioglycerol (Sigma, St. Louis, Mo.), 50 U/ml penicillin, and 50 μg/ml streptomycin, either in ultra-low attachment plates for the formation of suspended embryoid bodies (EBs) as previously described (Proc Natl Acad Sci USA, 2002. 99(7): p. 4391-6 and Stem Cells, 2007. 25(2): p. 392-401). Briefly, hES cells cultured on Matrigel® coated plate with conditioned media are treated by 2 mg/ml dispase (Invitrogen, Carlsbad, Calif.) for 15 minutes at 37° C. to loosen the colonies. The colonies are then scraped off, and transferred into ultra low-attachment plates (Corning Incorporated, Corning, N.Y.) for embryoid body formation.

### Example 8: Selection of Auxotrophic Regulatable Cell Lines

After using the CRISPR/Cas9 platform to generate various gene knockout cell lines. The cells are grown in defined, serum free media, and screened for lack of growth in the media without specific nutrient supplement. Kill curves with different concentrations of supplement vs. control are generated to demonstrate that an exogenously supplied version of the product of the knocked-out gene rescues the auxotrophic phenotype of the cell line. The most promising candidate gene knockouts are engineered in other commonly used cell lines to confirm the effect is universal. CRISPR/Cas9 technology is used to rapidly generate the knockout cell lines. Cell lines with multiple knockouts and mutations may be also generated to provide rapid multiplexed genome engineering and selection (e.g. 5 auxotrophic mutations and 5 antibiotics).

### Example 9: In Vivo Analysis

In vitro validated auxotrophic knockout cell lines may be analyzed in vivo. These cell lines are constrained by toxicity and bioavailability of the auxotrophic factor in humans. The gene knockout cell lines are engineered from human T-cells or any other lymphocyte. Conditional in vitro growth by the cell line is demonstrated in the presence of the auxotrophic factor, and not in the absence of the auxotrophic factor. The cell lines, e.g. human lymphocytes including an auxotrophic response system or element, confirmed to be auxotrophic for the factor may be administered in a mouse model. Only mice consuming the auxotrophic factor supplement are hypothesized to sustain growth of human lymphocytes including the auxotrophic gene deletion that is the auxotrophic response system or element. Further, cell growth stops in vivo upon removal of nutrient from the mouse food source.

1. A regulatable human cell line comprising an engineered genome, engineered to comprise or encode an auxotrophic response system, wherein the expression or activity of a gene has been knocked out or downregulated inducing auxotrophy in the regulatable human cell line.

2. The regulatable human cell line of claim 1, wherein the auxotrophic response system or element is responsive to the presence of an auxotrophic factor selected from the group consisting of a nutrient, an enzyme, an altered pH, an altered temperature, a non-organic molecule, a non-essential amino acid, an altered concentration of a moiety, and a niche environment.

3. The regulatable human cell line of claim 2, wherein under normal physiological conditions the nutrient or the enzyme is neither toxic nor bioavailable in concentrations sufficient to sustain the regulatable cell line in humans.

4. The regulatable human cell line of claim 2, wherein the nutrient is selected from the group of genes consisting of: uracil, biotin, nicotinic acid, glutamate(l−), lysine, adenine, ergosterol, glutamic acid, methionine, ethanolamine, dTMP, threonine, Cysteine, L-arginine, D-mannose, praline, arginine, valine, leucine, isoleucine, histidine, heme, leucine, oleic acid, 0.1 mM beta-alanine, methionine, lysine, valine, isoleucine, pantothenic acid, guanine, 0.25 mM spermine, riboflavin, D-glucosamine, 0.25 mM putrescine, thiamine, thiamine(l+), serine, 5-formyltetra hydrofolic acid, glutathione, galactose, nicotinic acid, ornithine, glutamine, 10 uM spermidine, tryptophan, and glutamic acid.

5. The regulatable human cell line of claim 2, wherein the nutrient is uracil or biotin.

6. The regulatable human cell line of claim 1, wherein the gene that has been knocked out or downregulated is selected from the group of genes consisting of: IBA57, AACS, AADAT, AASDHPPT, AASS, AC01, AC02, ACAT1, ACCS, ACCSL, ACO2, ACSS3, ADSL, ADSS, ADSSL1, ALAD, ALAS1, ALAS2, ALDH1A1, ALDH1A2, ALDH1A3, ALDH1B1, ALDH2, AMD1, ASL, ASS1, ATF4, ATFS, AZIN1, AZIN2, BCAT1, BCAT2, CAD, CBS, CBSL, CCBL1, CCBL2, CCS, CEBPA, CEBPB, CEBPD, CEBPE, CEBPG, CH25H, COO6, CPS1, CTH, CYP51A1, DECR1, DHFR, DHFRL1, DHODH, DHRS7, DHRS7B, DPYD, DUT, ETFDH, FAXDC2, FDFT1, FDPS, FDXR, FH, FPGS, G6PD, GCAT, GCH1, GCLC, GFPT1, GFPT2, GLRXS, GLUL, GMPS, GPT, GPT2, GSX2, H6PD, HAAO, HLCS, HMBS, HMGCL, HMGCLL1, HMGCS1, HMGCS2, HOXA1, HOXA10, HOXA11, HOXA13, HOXA2, HOXA3, HOXA4, HOXA6, HOXA7, HOXA9, HOXAS, HOXB1, HOXB10, HOXB11, HOXB12, HOXB13, HOXB2, HOXB3, HOXB4, HOXB5, HOXB6, HOXB7, HOXB8, HOXB9, HOXC4, HOXC5, HOXC6, HOXC7, HOXC8, HOXC9, HOXD1, HOXD10, HOXD11, HOXD12, HOXD13, HOXD3, HOXD4, HOXD8, HOXD9, HRSP12, HSD11B1, HSD11B1L, HSD17B12, HSD17B3, HSD17B7, HSD17B7B2, HSDL1, HSDL2, ICA1, ICA1P1, ID01, ID02, IL411, ILVBL, IP6K1, IP6K2, IP6K3, IPMK, IREB2, ISCA1, ISCA2, KATNA1, KATNAL1, KATNAL2, KDM1B, KDSR, KMO, KYNU, LGSN, LSS, MARS, MARS2, MAX, MITF, MLX, MMS19, MPC1, MPC1L, MPI, MSM01, MTHFD1, MTHFD1L, MTHFD2, MTHFD2L, MTHFR, MTRR, MVK, MYB, MYBL1, MYBL2, NAGS, ODC1, OTC, PAICS, PAOX, PAPSS1, PAPSS2, PDHB, PDX1, PFAS, PIN1, PLCB1, PLCB2, PLCB3, PLCB4, PLCD1, PLCD3, PLCD4, PLCE1, PLCG1, PLCG2, PLCH1, PLCH2, PLCL1, PLCL2, PLCZ1, PM20D1, PPAT, PSAT1, PSPH, PYCR1, PYCR2, PYCRL, QPRT, RDH8, RPUSD2, SCD, SCDS, SLC25A19, SLC25A26, SLC25A34, SLC25A35, SLC7A10, SLC7A11, SLC7A13, SLC7A6, SLC7A7, SLC7A8, SLC7A9, SLC7AS, SMOX, SMS, SNAPC4, SOD1, SOD3, SOLE, SRM, TAT, TFE3, TFEB, TFEC, THNSL1, THNSL2, TKT, TKTL1, TKTL2, UMPS, UROD, UROS, USF1, USF2, VPS33A, VPS33B, VPS36, VPS4A, and VPS4B.

7. The regulatable human cell line of claim 6, wherein the gene that has been knocked out or downregulated is uridine monophosphate synthetase (UMPS) or holocarboxylase synthetase (HLCS).

8. The regulatable human cell line of claim 1, wherein the regulatable cell line comprises at least one cell type selected from the group consisting of lymphocytes, induced pluripotent stem (iPS) cells, embryonic stem cells, somatic stem cells, haematopoetic stem cells, and peripheral blood mononuclear cells (PBMCs).

9. The regulatable human cell line of claim 1, wherein the regulatable human cell line is a T-cell line.

10. The regulatable human cell line of claim 1, further comprising the engineered genome encoding a therapeutic product that targets at least one selected from the group consisting of a cytokine, an antigen, and a stem cell.

11. The regulatable human cell line of claim 2, wherein the engineered genome comprises a knockout of a gene that encodes a protein that produces or metabolizes the auxotrophic factor.

12. A pharmaceutical composition comprising a regulatable human cell line of claim 1 and a pharmaceutically acceptable excipient.

13-16. (canceled)

17. A method of treating a disease, a disorder, or a condition in a subject, comprising (a) administering to the subject a regulatable human cell line according to claim 1, and (b) administering the auxotrophic factor to the subject in an amount sufficient to promote growth of the regulatable human cell line.

18-21. (canceled)

22. The method of claim 17, wherein the disease, the disorder, or the condition is selected from the group consisting of cancer, Parkinson's disease, graft versus host disease (GvHD), autoimmune conditions, hyperproliferative disorder or condition, malignant transformation, liver conditions, genetic conditions, juvenile onset diabetes mellitus, and ocular compartment conditions.

23. The method of claim 17, wherein the disease, the disorder, or the condition affects at least one system of the body selected from the group consisting of muscular, skeletal, circulatory, nervous, lymphatic, respiratory endocrine, digestive, excretory, and reproductive systems.

24. The method of claim 17, wherein the regulatable human cell line is regenerative.

25. The method of claim 17, further comprising contacting the subject with more than one regulatable human cell line.

26. The method of claim 17, further comprising contacting the subject with more than one auxotrophic factor.

27. The method of claim 17, wherein step (b) comprises localized release of the nutrient or the enzyme.

28. The method of claim 27, wherein the localized release is effected via utilization of a biocompatible device.

29. The method of claim 28, wherein the biocompatible device restricts diffusion of the regulatable human cell line in the subject.

30. The method of claim 17, further comprising removing the auxotrophic factor to deplete therapeutic effects of the regulatable cell line in the subject or to induce cell death in the regulatable human cell line.

31. The method of claim 17, wherein the therapeutic effects comprise at least one selected from the group consisting of: molecule trafficking, inducing cell death, recruitment of additional cells, and cell growth.

32. (canceled)

33. The method of claim 17, wherein the regulatable human cell line is derived from the subject prior to step (a).

34. (canceled)

35. A method of generating a regulatable human cell line according to claim 1, the method comprising the steps of:
(a) obtaining a pool of cells,
(b) knocking out or downregulating expression of a target gene selected from Table 1 by using a nuclease to introduce an addition, deletion or substitution in a target gene, and
(c) screening for auxotrophy.

36. (canceled)

37. The method of claim 35, wherein the target gene is selected from uridine monophosphate synthetase or holocarboxylase synthetase.

38. The method of claim 35, wherein the regulatable human cell line includes at least one type of cell selected from the group consisting of: lymphocytes, induced pluripotent stem (iPS) cells, embryonic stem cells, somatic stem cells, haematopoetic stem cells, and peripheral blood mononuclear cells (PBMCs).

39. The method of claim 35, wherein the nuclease is a CRISPR/Cas nuclease.

40. The method of claim 35, wherein the regulatable human cell line is auxotrophic for a factor listed in Table 1.

41. The method of claim 35, wherein the regulatable human cell line is auxotrophic for uracil or biotin.
